SEQUENCE LISTING (2 sequences)

SEQ ID NO: 1 - protein, 6 aa, Artificial Sequence
  Description of Artificial Sequencepeptide with modified residues
XRAXAX

SEQ ID NO: 2 - protein, 6 aa, Artificial Sequence
  Description of Artificial Sequencepeptide with modified residues
XXAXXX

(19) United States
(12) Patent Application Publication
(10) Pub. No.: US 2004/0101864 A1
(43) Pub. Date: May 27, 2004

(54) Chemical process

(75) Inventors: Nigel Phillip Taylor, Cheshire (GB); Kevin William Leslie, Cheshire (GB); Phillip John Hogan, Cheshire (GB); Francis Joseph Montgomery, Cheshire (GB); Edward John Bush, Cheshire (GB); Kay Alison Boardman, Cheshire (GB); Claire Ingrid Pulling, Cheshire (GB); Alan Charles Barker, Cheshire (GB)

Correspondence Address: ROPES & GRAY LLP, ONE INTERNATIONAL PLACE, BOSTON, MA, 02110-2624, US

(73) Assignee: AtraZeneca AB, Sodertalje (SE)

(21) Appl. No.: 10/333,571

(22) PCT Filed: Jul. 18, 2001
(86) PCT No.: PCT/GB01/03228

(30) Foreign Application Priority Data
Jul. 22, 2000 (GB) 0017979.6

(51) Int. Cl. C12Q 1/68; G01N 33/53; C07K 14/705; C07K 16/28; A61K 48/00; A61K 38/17

(52) U.S. Cl. 435/6; 530/350; 530/388.22; 435/7.1; 514/44; 514/12

(57) ABSTRACT

A process for the preparation of a salt of 5-phenylpentanoyl-(S)-arginyl-(S)-alanyl-{(S)-2-[(R)-3-amino-2-oxopyrrolidin-1-yl]propionyl}-(S)-alanyl-(S)-arginyl-(S)-alanyl-4-aminophenylacetamide (SEQ ID NO: 1) which comprises deprotection of a compound of the formula II or a salt thereof: wherein Pg and R¹ are defined in the description. Also claimed are intermediates used in the process and the processes for the preparation of the intermediates.

The invention concerns a novel chemical process, and more particularly it concerns a novel chemical process for the manufacture of salts of 5-phenylpentanoyl-(S)-alanyl-(S)-arginyl-(S)-alanyl-{(S)-2-[(R)-3-amino-2-oxopyrrolidin-1-yl]propionyl}-(S)-alanyl-(S)-arginyl-(S)-alanyl-4-aminophenylacetamide of the formula I (SEQ ID NO:1).

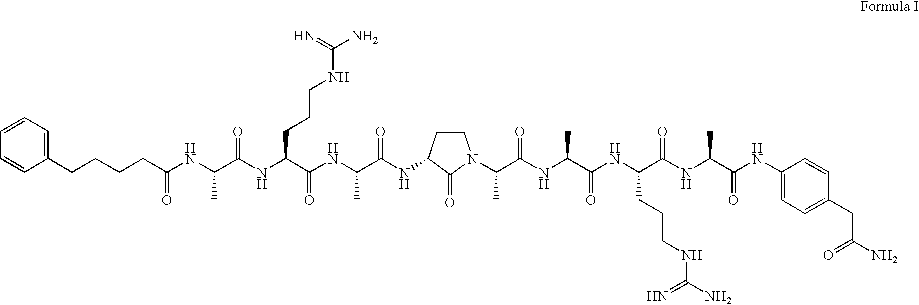

The pharmaceutically acceptable salts of the compound of formula I are disclosed in International Patent Application, Publication No. WO 97/31023 and possess pharmacologically useful properties for use in treating autoimmune diseases or medical conditions, such as rheumatoid arthritis and other MHC Class II dependent T-cell mediated diseases. WO 97/31023 discloses their preparation using solid phase synthesis, that is using a polymeric support to build up the molecule and subsequent cleavage of the molecule from the support. However the use of solid phase synthesis methodology is inconvenient and difficult when large scale manufacture is required. There is therefore a need to find an alternative procedure which avoids solid phase synthesis and which allows convenient and economic manufacture of the salts in a pure form. It is also particularly desirable for large scale manufacture to find a procedure which involves starting materials and intermediates which possess physical characteristics which allow them to be readily isolated in a pure form and in a good yield.

A process has now been discovered which does not involve solid phase synthesis and which is particularly advantageous for the manufacture of the salts of the compound of formula I. In one embodiment, the invention concerns a process for the manufacture of a salt of he compound of formula I which comprises deprotection of a compound of the formula II or a salt thereof:

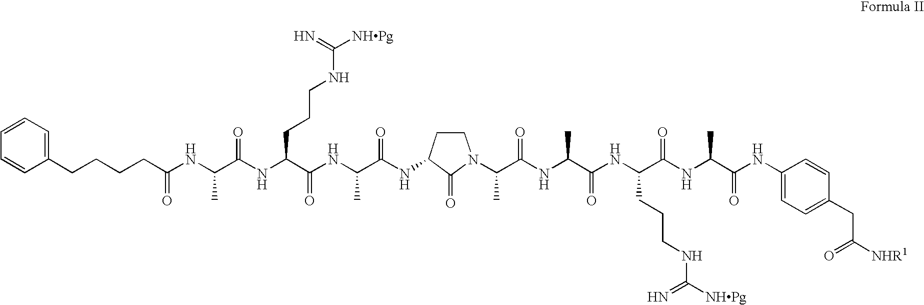

wherein: each Pg, independently, is an arginine protecting group; and R¹ is hydrogen or a protecting group for an amino group of an acetamide moiety.

It will be appreciated that salts obtained by this process which are not pharmaceutically acceptable salts are nevertheless useful for conversion to pharmaceutically acceptable salts by carrying out a subsequent salt exchange procedure. Such salt exchange procedures are well known in the art. Suitable salt exchange procedures include, for example an ion exchange technique, optionally followed by purification of the resultant product (for example by reverse phase liquid chromatography or reverse osmosis). Preferably the process is carried out so that the desired pharmaceutically acceptable salt is obtained directly without the need for a subsequent salt exchange procedure.

Pg may be any protecting group known in the art to be useful for the protection of a guanidino group in an arginyl residue. When R¹ is a protecting group for an amino group of an acetamide moiety it may be any protecting group known in the art to be useful for the protection of such a group. Suitable examples of protecting groups Pg and R¹ and conditions for their removal are disclosed, for example, in J Jones, The Chemical Synthesis of Peptides, Clarendon Press, Oxford, 1994; T Greeve, P Wuts, Protective Groups in Organic Synthesis, J Wyley & Sons, 3^{rd} Edition, 1999; and Bodanszky and Bodanszky, The Practice of Peptide Synthesis, Springer, 2^{nd} Edition, 1994. the disclosures of which are hereby incorporated by reference. It will be appreciated that the protecting groups Pg on the two arginyl residues may be the same or different, though preferably they are the same. A particularly preferred value for Pg is nitro. A particular value for R¹ when it is a protecting group is, for example benzyl. Preferably both Pg are nitro and R¹ is hydrogen. A particular advantage of using the compound of formula II wherein both Pg are nitro and R¹ is hydrogen is that, although this compound is amorphous, it can be obtained in a high state of purity by re-precipitation, for example by addition of aqueous acetone to a solution of the compound in DMF. A further advantage of using this particular formula II compound is that it can be obtained using intermediates which are themselves able to be isolated in a good yield and in a pure form.

When both Pg groups are nitro and R¹ is hydrogen, the nitro groups protecting the arginyl residues are preferably removed by chemical reduction, for example using catalytic hydrogenation, catalytic transfer hydrogenation or dissolving metal reductions such as zinc/acetic acid or tin/acetic acid. Catalytic hydrogenation is especially preferred. A suitable catalyst for catalytic hydrogenation includes, for example, palladium on charcoal, platinum oxide, palladium black and palladium salts such as Pd(II) acetate. The catalytic hydrogenation is conveniently carried out in the presence of a solvent or mixture of solvents. The choice of solvent or mixture of solvents may depend on whether a particular salt of the compound of formula I is desired. Suitable solvents include, for example, aqueous acetic acid, aqueous trifluoroacetic acid, aqueous formic acid or aqueous mineral acid, and especially aqueous acetic acid. The use of aqueous acetic acid (preferably in the ratio of acetic acid to water of 25:1 to 3:1 v/v, more preferably from 20:1 to 3:1v/v, or alternatively, in the ratio of acetic acid to water of from 1:3 to 3:1 v/v, for example 1:2 v/v) is particularly useful as the diacetate salt of the compound of formula I is formed directly, which is a particularly preferred salt. In a preferred embodiment the solvent comprises aqueous acetic acid and a second acid which is stronger than acetic acid. The second acid has a pKa which is lower than that of acetic acid. Suitable second acids include mineral acids or more preferably organic acids, such as a fluorinated acetic acid, for example di- or tri-fluoroacetic acid. Preferably an excess of the acetic acid is present relative to the second acid (for example a ratio of acetic acid to second acid of from 2:1 to 40:1 v/v, more preferably from 5:1 to 30:1 v/v). In this embodiment the second acid is preferably present in an equimolar, or more preferably at a molar excess relative to the compound of Formula II, for example from 1 to 10, more preferably from 2 to 8 molar equivalents of the second acid relative to the compound of formula II. A particularly useful solvent includes, for example, aqueous acetic acid containing 5 equivalents of trifluoroacetic acid per equivalent of the compound of formula II. A particularly preferred catalyst for catalytic hydrogenation includes 3-20% palladium on charcoal, for example 5-10% palladium on charcoal, or palladium on zeolite or silica. The catalysts are preferably used in an amount such that there is 0.3 to 1.2% w/w palladium per compound of formula II or salt thereof. The hydrogenation is preferably carried out at a hydrogen pressure of 0-100 bar gauge, and preferably at 0-10 bar gauge and especially from 1 to 5 bar gauge. Conveniently the catalytic hydrogenation is carried out at a temperature in the range of, for example, 10-70° C., preferably 20-50° C.

Pharmaceutically acceptable salts include, for example, salts with acids forming physiologically acceptable anions, such as salts with mineral acids, for example, hydrogen halides (such as hydrogen chloride and hydrogen bromide), sulfonic and phosphonic acids, and with organic acids such as acetic acid, oxalic acid, tartaric acid, p-toluenesulfonic acid, methanesulfonic acid, trifluoroacetic acid and the like.

In a second embodiment, the invention concerns a process for the manufacture of a compound of formula II or a salt thereof, which comprises coupling a carboxylic acid of the formula III or a salt thereof,

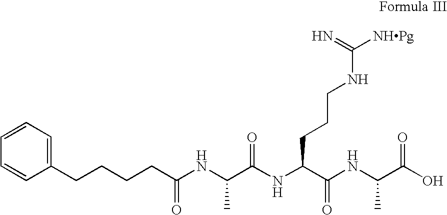

wherein Pg is as defined above (and is preferably nitro) with an amine of the formula IV

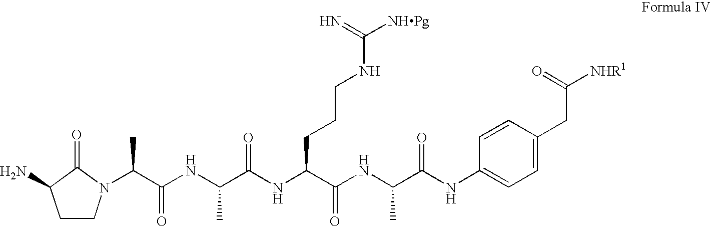

wherein Pg and R¹ are as defined above (and preferably Pg is nitro and R¹ is hydrogen).

The coupling reaction is carried out using any standard procedure known in the art for coupling acids with amines to form amides. Such procedures are, for example, described in Bodansky and Bodansky (supra), the disclosures of which are incorporated herein by reference. In particular, for example, the coupling is suitably carried out in an organic solvent such as N,N-dimethylformamide (DMF), dichloromethane (DCM), N-methylpyrrolidinone (NMP) or tetrahydrofuran (THF) in the presence of a coupling reagent. Typical coupling reagents include, for example, dicyclohexylcarbodiimide (DCCI), diisopropylcarbodiimide (DIC) or 1-(3-dimethylaminopropyl-3-ethylcarbodiimide (EDCI) in the presence of 1-hydroxybenzotriazole (HOBt), or 2-(1H-benzotriazol-1-yl)-1,1,3,3-tetramethyluronium tetrafluoroborate, in the presence of a tertiary amine base such as N-methylmorpholine (NMM) or diisopropylethylamine (DIPEA). Preferably EDCI and HOBt in the presence of NMM are used. When EDCI is used as the coupling agent it is preferably in the form of the hydrochloride addition salt. When the coupling is performed in the presence of HOBt, the HOBt is preferably used in the form of its monohydrate.

Typically the coupling is initially carried out at low temperature, for example in the range of −5° C. to +5° C., and the reaction mixture can be allowed to attain ambient temperature. In a preferred embodiment the coupling is performed in DMF or NMP at a temperature of less than 0° C., for example in the range of from 0 to −5° C. It is especially preferred that the coupling is performed in DMF at a temperature in the range of from 0 to −5° C. A further embodiment of the invention is a process for the manufacture of a salt of a compound of the formula I which comprises coupling a carboxylic acid of the formula III or a salt thereof as defined above with an amine of the formula IV as defined above to form a compound of the formula II or a salt thereof, followed by deprotection of the compound of the formula II or salt thereof wherein Pg is an arginine protecting group and R¹ is hydrogen or a protecting group for an amino group of an acetamide moiety (such as benzyl) to form a salt of a compound of the formula I. Preferably in this process, Pg is nitro and R¹ is hydrogen.

Preferably the compound of formula IV is generated from a protected form thereof, for example by using a compound of the formula V

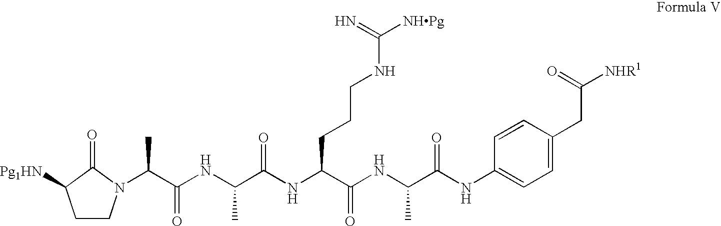

wherein Pg is as defined above (and is preferably nitro), R¹ is as defined above (and is preferably hydrogen), and Pg₁ is an amino protecting group. It is important that Pg₁ is chosen such that it can be selectively removed in the presence of Pg and R¹ if the latter is other than hydrogen. When Pg is nitro and R¹ is hydrogen or benzyl, the protecting group Pg₁ is preferably one which can be readily removed under acidic conditions, such as a tert-butyloxycarbonyl (Boc) group. This protecting group can then be removed using, for example, hydrogen chloride gas or an aryl sulphonic acid. Suitable aryl sulphonic acids include, for example toluene sulphonic acid or, more preferably, benzene sulphonic acid. It is especially preferred that benzene sulphonic acid is used to remove Pg₁ when it is Boc. The removal of Pg₁ is preferably carried out in an inert solvent. Suitable inert solvents include, for example dichloromethane, tetrahydrofuran or ethyl acetate. If desired the solvent can be exchanged for another solvent such as DMF or NMP prior to carrying out the coupling reaction without further purification of the compound of formula IV formed. Other suitable values for Pg, R¹ and Pg₁ which allow selective removal of Pg₁ in the presence of Pg and R¹ if the latter is other than hydrogen are well known in the art.

Accordingly a preferred aspect of the present invention comprises a process for the manufacture of a salt of the compound of the formula I which comprises the steps of

(1) selective removal of an amino protecting group Pg. from a compound of the formula V (preferably Pg₁ is Boc) wherein Pg and R¹ are as defined above (preferably Pg is nitro and R¹ is preferably hydrogen or benzyl and especially hydrogen) to form an amino compound of the formula IV as defined above;

(2) coupling the amino compound of the formula IV with a carboxylic acid of the formula III or a salt thereof as defined above to form a compound of the formula II or a salt thereof as defined above; and

(3) deprotection of the compound of the formula II or a salt thereof wherein Pg is an arginine protecting group and R¹ is hydrogen or a protecting group for an amino group of an acetamide moiety (such as benzyl) to form a salt of a compound of the formula I.

Preferably in this process, Pg is nitro and R¹ is hydrogen.

Preferably a compound of formula III or a salt thereof as defined above is prepared by hydrolysis of an ester of formula VI

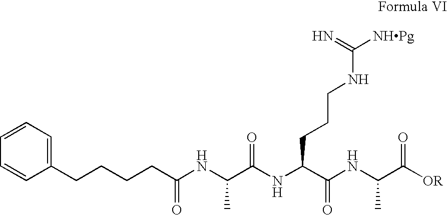

wherein R is alkyl for example (1-6C)alkyl, or aralkyl (for example phenyl(1-6C)alkyl such as benzyl) and Pg is as defined above (preferably nitro). Typically the hydrolysis is carried out under aqueous base conditions, for example using an aqueous solution of an alkali metal hydroxide (such as sodium hydroxide or lithium hydroxide) and a suitable organic solvent (such as acetonitrile) The hydrolysis is conveniently carried out at ambient temperature. The reaction mixture is subsequently acidified, for example using hydrochloric acid, to give the free acid. It is particularly advantageous to use the compound of formula VI wherein R is methyl and Pg is nitro, as it has been found that this compound can be obtained in crystalline form. This finding is particularly advantageous as it greatly assists in obtaining a salt of the compound of formula I in a pure and uniform form. The compound of formula VI may be crystallised by attaining a supersaturated solution of the compound of formula VI. The formation of a supersaturated solution may be achieved using known techniques for example by cooling a solution of the compound in a suitable solvent, evaporating solvent from a solution of the compound, or by the addition of an anti-solvent to a solution of the compound of formula VI, wherein the anti solvent is one in which the compound of formula IV is insoluble or sparingly soluble. Suitable solvents for the crystallisation of the compound of formula VI include acetonitrile, butyronitrile, isobutanol or ethylacetate. Preferably the compound of formula VI wherein Pg is nitro and R is methyl is crystallised from acetonitrile.

Accordingly a further preferred aspect of the present invention comprises a process as defined in steps (1), (2) and (3) above wherein the compound of formula III used in step (2) is obtained by hydrolysis of a compound of the formula VI wherein R is as defined above (preferably methyl), and Pg is as defined above (preferably nitro).

A compound of the formula V is obtained by coupling a compound of the formula VII wherein Pg and R¹ are as defined above (preferably Pg is nitro and R¹ is hydrogen)

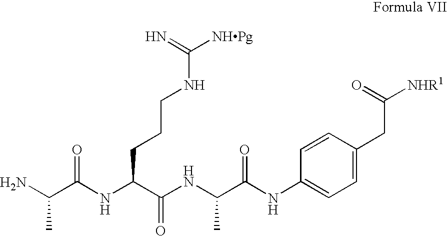

with a carboxylic acid of the formula VIII or a salt thereof

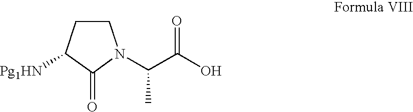

wherein Pg₁ is an amino protecting group, preferably Boc.

The conditions for carrying out this coupling reaction are analogous to those described above for coupling the compounds of formula III and IV. A mixture of acetonitrile and DMF is a preferred solvent mixture for use in this coupling reaction. Preferably the temperature during this coupling reaction is 0° C. or less, more preferably from 0 to −10° C. and especially from 0 to −5° C.

Alternatively a compound of the formula V is obtained by coupling a compound of the formula XI

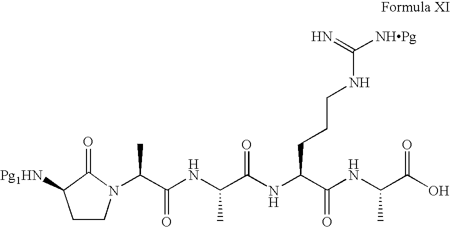

wherein Pg and Pg₁ are as defined above (preferably Pg is nitro and Pg₁ is Boc) with a compound of the formula XII

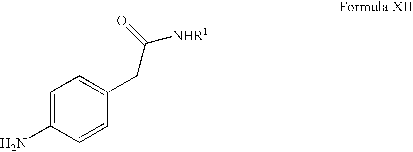

wherein R¹ is hydrogen or a protecting group, for example benzyl.

A further aspect of the invention therefore comprises a process as defined in steps (1), (2) and (3) above wherein the compound of formula V used in step (1) is obtained by coupling a compound of the formula VII wherein Pg and R¹ are as defined above (preferably Pg is nitro and R¹ is hydrogen) with a carboxylic acid of the formula VIII or a salt thereof wherein Pg₁ is an amino protecting group capable of being selectively removed in the presence of Pg and R¹, and is preferably Boc.

A further aspect of the invention comprises a process as defined in steps (1), (2) and (3) above wherein the compound of formula V used in step (1) is obtained by coupling a compound of the formula XII wherein R¹ is as defined above (preferably hydrogen or benzyl) with a compound of the formula XI wherein Pg and Pg₁ are as defined above (preferably Pg is nitro and Pg₁ is Boc).

A compound of formula VII is preferably obtained by selectively removing the amino protecting group Pg₂ from a compound of formula IX

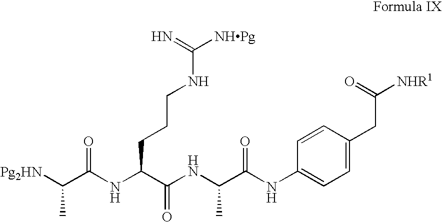

wherein Pg and R¹ are as defined above (preferably Pg is nitro and R¹ is hydrogen or benzyl, preferably hydrogen), and Pg₂ is an amino protecting group which can selectively removed in the presence of Pg and R¹ if the latter is other than hydrogen. Pg₂ is preferably one of the preferred amino protecting groups mentioned above in relation to Pg₁, more preferably Pg₂ is Boc which may be removed under mild acidic conditions as described above. When Pg₂ is Boc it is preferably removed using toluene sulphonic acid or more preferably benzene sulphonic acid.

The compounds of formula VIII wherein Pg₁ is Boc may be obtained as described in the examples hereinafter and other compounds of formula VIII may be made by analogy therewith. A suitable process for preparing the compound of the formula VIII comprises, for example, hydrolysis of the ester of the formula Villa

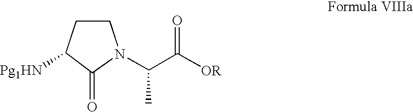

wherein Pg₁ is as defined above (preferably Boc) and R is alkyl, for example (1-6C)alkyl, or aralkyl (for example phenyl(1-6C)alkyl such as benzyl)(1-6C)alkyl. Preferably R is (1-6C)alkyl, more preferably (1-4C)alkyl and especially methyl.

The hydrolysis may typically be carried out using similar conditions to those described above for the hydrolysis of a compound of the formula VI.

In a preferred embodiment the hydrolysis of the compound of formula VIIIa is performed under aqueous basic conditions using lithium hydroxide as the base. The hydrolysis is preferably carried out at a temperature in the range of from 0 to 10° C., more preferably from 0 to 50° C.

The compound of formula VIIIa may be prepared using known methods, for example as described in Example 1 of WO 97/31023 or by the process described in WO 99/55669. Alternatively, we have found that the compound of formula VIIIa may be prepared by an analogous process to those described above but using an alternative methylating agent, for example dimethylsulfate.

A compound of the formula XI may be obtained, for example, by hydrolysis of the corresponding ester of formula XIII

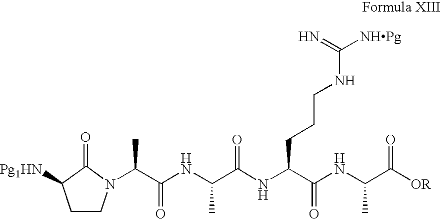

wherein Pg and Pg₁ are as defined above and R is alkyl (for example (1-6C)alkyl or preferably 1-4C)alkyl) or aralkyl (for example phenyl(1-6C)alkyl such as benzyl). The hydrolysis may typically be carried out using similar conditions to those described above for the hydrolysis of a compound of the formula VI.

A particular advantage of using a compound of the formula IX or XI, and a compound of the formula VI, in the processes described above for manufacturing a compound of the formula I is that these compounds can be obtained from the same starting material of formula X

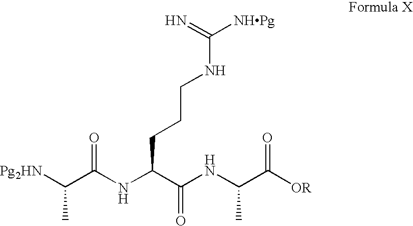

wherein Pg, Pg₂ and R are as defined above. This has the advantage of reducing the number of process stages required to prepare the compound of formula (I).

Preferably in formula X, Pg is nitro, Pg₂ is Boc and R is methyl, as it has surprisingly been found that this compound is crystalline and can, therefore be prepared in a pure form. This compound can be crystallised from a suitable solvent using analogous methods to those described above for the crystallisation of the compound of formula VI. Suitable solvents for crystallising the compound include, for example acetonitrile.

A compound of the formula VI can be obtained from a compound of formula X by removal of Pg₂ and coupling with 5-phenylpentanoic acid. In a preferred embodiment the coupling is performed in the presence of methanol, more preferably in a mixture of methanol and DCM. Preferably the compound of formula VI is isolated in a crystalline form, as described above.

A compound of the formula IX may be obtained from a compound of formula X by hydrolysis of the ester functionality to form a carboxylic acid group and coupling the compound thus formed with a compound of formula XII. The hydrolysis and coupling reactions may be carried out using analogous processes to those described above. A preferred solvent for the hydrolysis and coupling reactions is THF.

A compound of the formula XI may be obtained from a compound of the formula X by removal of Pg₂, coupling with a compound of the formula VIII and hydrolysing the ester functionality to form a carboxylic acid. The hydrolysis may typically be carried out using similar conditions to those described above for the hydrolysis of a compound of the formula VI.

4-Aminophenylacetamide (formula XII, R¹ is H) may be obtained, for example, as described in the examples hereinafter. A preferred process for the preparation of 4-aminophenylacetamide comprises the steps:

(i) esterification of 4-aminophenylacetic acid with a suitable alcohol in the presence of sulphuric acid to give a 4-aminophenylacetate ester hydrogensulphate salt; and

(ii) reacting the product of step (i) with ammonia.

The alcohol used in step (i) is preferably a (1-4C)alkanol for example ethanol or, more preferably, methanol. A suitable reaction temperature for step (i) is less than 30° C., more preferably less than 25° C. Step (ii) of this process is preferably carried out in an aqueous medium, more preferably in water containing dissolved sodium chloride. Preferably aqueous ammonia is added to an aqueous solution of the product of step (i). Preferably the product of step (i) is isolated in a crystalline form prior to step (ii) of the process. The product of step (i) may be crystallised from a suitable solvent, for example from methyl tert-butyl ether. We have found that this preferred process provides 4-aminophenylacetamide in high yield and in pure form. This preferred process is a further aspect of the present invention.

A compound of the formula XII in which R¹ is a protecting group, such as benzyl, may be obtained for example by removal of the amino protecting group Z from the compound of the formula XIIa, wherein Z is an amine protecting group as hereinbefore defined for Pg₁ (for example Boc):

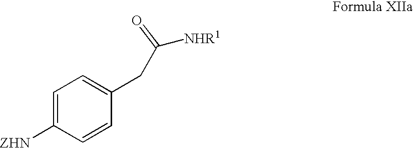

wherein R¹ is as hereinbefore defined. The protecting group Z may be removed using analogous conditions to those described above for the removal of Pg₁.

The compound of the formula XIIa may be prepared for example by coupling the compound of the formula XIIb with a compound of the formula R¹NH₂ wherein R¹ is as hereinbefore defined:

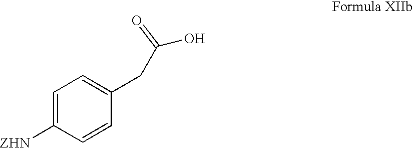

wherein Z is as defined above.

For example, when R¹ in the compound of formula XII is benzyl this compound may be prepared by coupling 4-(butoxycarbonylamino)phenylacetic acid with benzylamine, followed by removal of the Boc group under acidic conditions. Analogous coupling conditions to those described above for the coupling of compounds of formula III and IV may be used in these cases. A suitable solvent for this coupling reaction includes, for example tetrahydrofuran.

A compound of the formula X may be obtained by selective removal of Pg₃ from a compound of formula XIV by and coupling with a Pg₂ protected (S)-alanine:

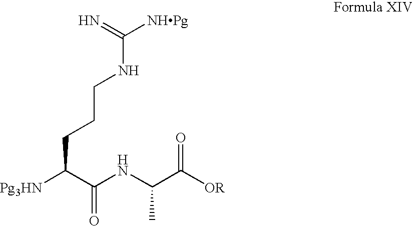

wherein Pg₃ is a suitable amino protecting group which can be selectively removed in the presence of Pg; and R, Pg and Pg₂ are as hereinbefore defined. Suitable groups represented by Pg₃ are as hereinbefore defined for Pg₂, preferably Boc. Preferably in formula XIV Pg is nitro nd Pg₃ is Boc As will be understood, Pg₂ in the Pg₂ protected (S)-alanine is a protecting group for the amine in the (S)-alanine. Preferably Pg₂ and Pg₃ are the same, more preferably Pg₂ and Pg₃ are both Boc. R is preferably (1-4C)alkyl, more preferably methyl.

The conditions for the removal of Pg₃ are analogous to those described above for the removal of Pg₁ from the compound of formula V. Following removal of Pg₃ from the compound of formula XIV, the conditions for the coupling with the Pg₂ protected (S)-alanine are analogous to those described above for the coupling of the compounds of formula III and IV. When Pg₃ is Boc it is preferably removed using an arylsulphonic acid, more preferably toluene sulphonic acid or, especially benzene sulphonic acid.

Preferably the compound of formula X is isolated in a crystalline form as described above.

A compound of formula XIV may be obtained from the coupling of a compound of the formula XV or a salt thereof, and a compound of the formula XVI or a salt thereof:

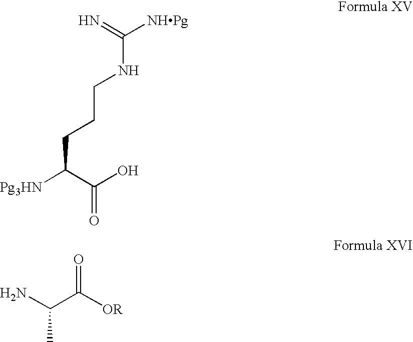

wherein Pg, Pg₃ and R are as hereinbefore defined (preferably Pg is nitro, Pg₃ is Boc and R is methyl). Suitable conditions for the coupling of the compounds of formula XV and XVI are analogous to those used for the coupling of the compounds of formulae III and IV described above.

Preferably the compound of formula XV is tert-butyloxycarbonyl-(S)-arginyl(NO₂)—OH. Preferably the compound of formula XVI is a alanine (1-6C)alkyl ester hydrochloride, more preferably alanine methyl ester hydrochloride.

We have surprisingly found that when Pg is nitro, Pg₃ is Boc and R is methyl the compound of formula XIV is crystalline. This compound may be isolated in a crystalline form by crystallisation from a suitable solvent, such as a (1-6C)alkylacetate, for example propyl acetate or n-butyl acetate.

It is preferred that the compound of formula XIV is isolated in crystalline form prior to coupling with the Pg₂ protected (S)-alanine to form the compound of formula X, because this minimises the formation of undesirable impurities. However, if desired the coupling of the compounds of formula XV and XVI followed by coupling with the Pg₂ protected (S)-alanine may be telescoped together.

Certain intermediates of the formula II, III, IV, V, VI, VII, VIII, IX, X , XI XII and XIII are novel and are further independent aspects of the invention. Further independent aspects of the invention are the processes described herein for preparing the novel intermediates.

The invention will now be illustrated by the following non-limiting examples in which, unless otherwise stated:-

(i) concentrations and evaporations were carried out by rotary evaporation in vacuo;

(ii) operations were carried out at room temperature, that is in the range 18-26° C.;

(iii) yields, where given, are intended for the assistance of the reader only and are not necessarily the maximum attainable by diligent process development;

(iv) ¹H NMR spectra were determined using tetramethylsilane (TMS) as an internal standard, and are expressed as chemical shifts (delta values) in parts per million relative to TMS using conventional abbreviations for designation of major peaks: s, singlet; d, doublet; m, multiplet; t, triplet; br, broad.

### EXAMPLE 1

### (Preparation of the Trifluoroacetate Salt of Compound of Formula I (SEQ ID NO:1) from a Compound of Formula II (Pg=nitro; R¹=H)

5-Phenylpentanoyl-(S)-alanyl-{(S)-arginyl(NO₂)-(S)-alanyl-((S)-2-[(R)-3-amino-2-oxopyrrolidin-1-yl]propionyl}-(S)-alanyl-(S)-arginyl(NO₂)-(S)-alanyl-4-aminophenylacetamide (formula II; Pg=nitro) (SEQ ID NO:2) (0.75 g) and 5% palladium on carbon (0.083 g of a 60% water wet paste, 6:4 dry solid to water) were added to a mixture of acetic acid (6 ml), water (2 ml) and trifluoroacetic acid (0.24 ml) in a glass pressure vessel and the mixture was purged three times with argon at a pressure of 4 bar. The mixture was heated to 50° C. and then purged with hydrogen at a pressure of 4 bar. The mixture was stirred at 50° C. and at a pressure of 5.5 bar for 3.25 hours. The pressure vessel was then purged three times with argon at a pressure of 4 bar. The reaction mixture was filtered hot through a water wet pad of diatomaceous earth. The vessel and cake were washed with water (2×4 ml). The combined filtrates were concentrated by evaporation to give a colourless oil. The oil was dissolved in methanol (25 ml) and the mixture concentrated by evaporation. This procedure was repeated several times to give a colourless oil (0.92 g). The oil was dissolved in hot methanol (5 ml) and ethyl acetate (20 ml) was added to precipitate a white solid. The solid was collected by vacuum filtration and washed with ethyl acetate (5 ml) to give 5-phenylpentanoyl-(S)-alanyl-(S)-arginyl-(S)-alanyl-{(S)-2-[(R)-3-amino-2-oxopyrrolidin-1-yl]propionyl}-(S)-alanyl-(S)-arginyl-(S)-alanyl-4-aminophenylacetamide of formula I (SEQ ID NO:1) as the ditrifluoroacetate salt (0.78 g);

¹H NMR (d₆-DMSO): 1.20 (m, 6H), 1.25 (d, 3H), 1.28 (d, 3H), 1.32 (d, 3H), 1.51 (m, 10H), 1.70 (m, 3H), 2.14 (m, 2H), 2.29 (m, 1H), 2.57 (t, 2H), 3.09 (m, 4H), 3.36 (m, 4H), 4.26 (m, 5H), 4.38 (m, 2H), 4.60 (dd, 1H), 7.13-7.30 (m, 7H), 7.50 (d, 2H). The ditrifluoroacetate salt of the compound of formula I may be converted to the diacetate salt by passing a solution of the ditrifluoroacetate salt through an ion exchange column in the presence of ammonium acetate. The resulting product may then be purified using reverse phase liquid chromatography.

### EXAMPLE 2

### (Preparation of Compound of Formula II (Pg=nitro; R¹=H) (SEQ ID NO:2) from Compound of Formula III (Pg=nitro) and compound of formula IV (Pg=nitro; R¹=H))

{(S)-2-[(R)-3-Amino-2-oxopyrrolidin-1-yl]propionyl}-(S)-alanyl-(S)-arginyl(NO₂)-(S)-alanyl-4-aminophenylacetamide (formula IV; Pg=nitro) (5.8 g) was dissolved in DMF (25 ml) with stirring at 21° C. The solution was cooled to −3° C. to −4° C. and N-methylmorpholine (1.5 ml) was added slowly maintaining the temperature of the mixture between −1° C. and −4° C. A white solid precipitated. When the addition was complete, the mixture was stirred and warmed to 21° C. to give a clear solution. 5-Phenylpentanoyl-(S)-alanyl-(S)-arginyl(NO₂)-(S)-alanine (formula III; Pg=nitro) (3.27 g) and 1-hydroxybenzotriazole monohydrate (0.63 g) were added and the mixture was stirred at 21° C. until a clear solution was obtained. The solution was then cooled to −4° C. and 1-(3-dimethylaminopropyl)-3-ethylcarbodiimide hydrochloride (1.48 g) was added in one portion. The reaction mixture was stirred at −4° C. for 4.75 hours. Cooling was stopped and water (48 ml) was added dropwise over 7 minutes. A mixture of acetone (48 ml) and water (48 ml) was added dropwise over 10 minutes and the reaction mixture was stirred for 16 hours at 21° C. to 25° C. The solid which precipitated was collected by vacuum filtration and washed with water (90 ml), then acetone (2×90 ml), to give 5phenylpentanoyl-(S)-alanyl-(S)-arginyl(NO₂)-(S)-alanyl-{(S)-2-[(R)-3-amino-2-oxopyrrolidin-1-yl]propionyl}-(S)-alanyl-(S)-arginyl(NO₂)-(S)-alanyl-4-aminophenylacetamide (formula II; Pg=nitro) (SEQ ID NO:2) (6.0 g);

¹H NMR (d₆-DMSO): 1.18 (d, 3H), 1.20 (d, 3H), 1.23 (d, 3H), 1.27 (d, 3H), 1.31 (d, 3H), 1.52 (m, 10H), 1.70 (m, 3H), 2.13 (t, 3H), 2.29 (m, 1H), 2.56 (t, 3H), 3.15 (m, 4H), 3.33 (m, 4H), 4.27 (m, 5H), 4.38 (m, 2H), 4.60 (dd, 1H), 7.13-7.29 (m, 7H), 7.51 (d, 2H).

Preparation of the Compound of Formula IV:

{(S)-2-[(R)-3-(N-[tert-butyloxycarbonyl]amino)-2-oxopyrrolidin-1-yl]propionyl}-(S)-alanyl-(S)-arginyl(NO₂)-(S)-alanyl-4-aminophenylacetamide (formula V, Pg=nitro; Pg¹=Boc) (5.0 g) was stirred in a saturated solution of hydrogen chloride in ethyl acetate (90 ml) at 22° C. to 24° C. for 3 hours. Argon was then bubbled for 30 minutes through the reaction mixture. The reaction mixture was then concentrated under vacuum. The resultant solid was triturated with and evaporated from ethyl acetate and collected by filtration to give {(S)-2-[(R)-3-amino-2-oxopyrrolidin-1-yl]propionyl}-(S)-alanyl-(S)-arginyl(NO₂)-(S)-alanyl-4-aminophenylacetamide (formula IV; Pg=nitro) (5.92 g).

### EXAMPLE 3

### (Preparation of Compound of Formula V (Pg=nitro; Pg₁=Boc; R¹=H) from Compound of Formula VII (Pg=nitro; R¹=H) and Compound of Formula VIII (Pg¹=Boc))

(S)-Alanyl-(S)-arginyl(NO₂)-(S)-alanyl-4-aminophenylacetamide (formula VII; Pg=nitro) (44.5 g), (S)-2-[(3R)-3-(N-[tert-butyloxycarbonyl]amino)-2-oxopyrrolidin-1-yl]propionic acid (formula VIII, Pg₁=Boc) (21.14 g) and 1-hydroxybenzotriazole monohydrate (5.93 g) were added to a mixture of acetonitrile (863 ml) and DMF (128 ml). The stirred mixture was cooled to −2° C. and N-methylmorpholine (20.2 ml) was added slowly. The temperature of the mixture rose to about 0° C. The mixture was re-cooled to −3° C. and 1-(3-dimethylaminopropyl)-3-ethylcarbodiimide hydrochloride (14.16 g) was added with stirring. The mixture was stirred for 20 hours at −3° C. to −5° C. and then concentrated to give an oil containing a fine white precipitate. The solid was removed by filtration, washing the solid with acetonitrile. The washings and filtrate were combined and concentrated under high vacuum to give an oil (132 g). Isobutanol (860 ml) was added to the oil and the mixture was washed successively with 10% aqueous sodium chloride solution, 1.0M sodium hydrogen sulfate solution, aqueous sodium carbonate solution and again with 10% sodium chloride solution. This washing procedure was repeated until the pH of the final wash with sodium chloride solution was 7. The organic phase was distilled, adding isobutanol at intervals, until the still head temperature reached 107° C. The solution was then filtered through a pad of diatomaceous earth in a jacketed filter (jacket temperature 65° C.). The filtered solution was reheated to reflux to give a clear solution. The solution was allowed to cool with stirring to 66° C., at which point stirring was stopped and the mixture allowed to cool to ambient temperature. The precipitated solid was collected by filtration, washed with isobutanol and dried to constant weight in a vacuum oven at 45° C. There was thus obtained -((S)-2-[(R)-3-[N-tert-butyloxycarbonyl]amino-2-oxopyrrolidin-1-yl]propionyl)-(S)-alanyl-(S)-arginyl(NO₂)-(S)-alanyl-4-aminophenylacetamide (formula V, Pg=nitro; Pg₁=Boc) (48.5 g);

¹H NMR (d₆-DMSO): 1.23 (d, 3H), 1.27 (d, 3H), 1.32 (d, 3H), 1.39 (s, 9H), 1.53 (m, 3H), 1.73 (m, 2H), 2.23 (m, 1H), 3.16 (m, 2H), 3.31 (m, 4H), 4.11 (m, 1H), 4.25 (m, 2H), 4.38 (m, 1H), 4.55 (m, 1H), 7.18 (m, 2H), 7.52 (m, 2H).

Preparation of the Compound of Formula VII:

A solution of hydrogen chloride in ethyl acetate (335 ml) was added to tert-butyloxycarbonyl-(S)-alanyl-(S)-arginyl(NO₂)-(S)-alanyl-4-aminophenylacetamide (formula IX, Pg=nitro; Pg₂=Boc) (43.2 g) cooled in an ice-water bath. The mixture was stirred for ten minutes with cooling, then allowed to rise to ambient temperature. After 7.5 hours, the mixture was filtered and the isolated solid was washed with ethyl acetate (4×125 ml). The solid was then dried under vacuum at ambient temperature for 16 hours to give (S)-alanyl-(S)-arginyl(NO₂)-(S)-alanyl-4-aminophenylacetamide (formula VII; Pg=nitro) (44.5 g) which was used without further purification.

Preparation of the Compound of Formula VIII (Pg₁=Boc):

Sodium hydroxide solution (1.88M; 150 ml) was added to (R)-methionine (25.0 g; 0.166 mol) and tert-butanol (100 ml) was added. The mixture was cooled to 0-5° C. and di-tert-butyl dicarbonate (41.1 g) added in one portion. The reaction mixture was warmed to 20° C. and stirred for 4 hours. The mixture was cooled to 0-5° C. and 2M aqueous citric acid solution (128 ml) was added, maintaining the temperature below 5° C. Dichloromethane (250 ml) was added and the mixture stirred at 20° C. for 15 minutes. The upper aqueous phase was separated and the organic phase retained. The aqueous phase was extracted with dichloromethane (125 ml) and the extract was combined with the retained organic phase. The combined organic phase was washed with water (250 ml) and distilled at atmospheric pressure until a volume of 250 ml remained. The solution (which contains Boc-(R)-methionine) was cooled to 0-5° C. and (S)-alanine methyl ester hydrochloride (25.7 g), 1-hydroxybenzotriazole hydrate (24.6 g), 1-(3-dimethylaminopropyl)-3-ethylcarbodiimide hydrochloride (35.6 g) and N-methyl-morpholine (35.6 g) was added maintaining the temperature of the mixture below 5° C. The mixture was then warmed to 20° C. and stirred at this temperature for 5 hours. The mixture was cooled to 0-5° C. and water (100 ml) was added, maintaining the temperature below 5° C., and the mixture was stirred for 15 minutes. The organic phase was separated and washed successively with water (150 ml), 2M aqueous citric acid solution (100 ml), 20% aqueous sodium bicarbonate solution (100 ml) and brine (100 ml). Dichloromethane (450 ml) was added to the organic phase and the mixture distilled at atmospheric pressure until 100 ml of distillate was collected. The mixture (which contains Boc-(R)-Met-(S)-Ala-OMe) was cooled to 0-5° C. and trimethyloxonium tetrafluoroborate (25.1 g; 0.166 mol) was added in one portion keeping the temperature at 0-5° C. The mixture was allowed to warm to 20° C. over 30 minutes and then stirred for a further 4 hours. Powdered potassium carbonate (325 mesh; 71.9 g) was added and the mixture was refluxed for 12 hours. The mixture was cooled to 0-5° C. and water (300 ml) was added. The mixture was stirred for 15 minutes at 20° C. and filtered through a sinter funnel (porosity 3). The lower organic phase of the filtrate was separated and washed with water (300 ml). The solution was distilled at atmospheric pressure until 320 ml of distillate was collected and n-butyl acetate (200 ml) was added. The solution was concentrated at 70-75° C. under reduced pressure until 80 ml of concentrate remained. The concentrate was cooled to 40° C. and isohexane (80 ml) was added. The mixture was cooled to 20° C., then heated to 40° C. and additional isohexane (320 ml) added slowly over 1 hour. The mixture was stirred a further 30 minutes at 40° C. and then cooled to 0-5° C. and stirred for 1 hour. The suspended crystalline solid was collected by filtration, washed with cold isohexane (2×50 ml), and dried at 50° C. in a vacuum oven for 8 hours to give methyl (S)-2-[(R)-3(N-[tert-butyloxycarbonyl]amino)-2-oxopyrrolidin-1-yl]propionate (36.5 g). The product (25 g) in water (195 ml) was cooled to 0-5° C. and sodium hydroxide in water (47% w/w; 5.45 ml) was added over one hour with stirring. When the addition was complete the reaction mixture was stirred for a further 90 minutes at 0-5° C. and a solution of potassium hydrogen sulfate (13.67 g) in water (50 ml) was then added to the cold mixture over 2 hours. After a further hour the cold mixture was filtered. The collected solid was washed with a small volume of water and dried under vacuum at 40° C. to give (S)-2-[(R)-3-(N-[tert-butyloxycarbonyl]amino)-2-oxopyrrolidin-1-yl]propionic acid (21.8 g).

Alternative Preparation of the Compound of Formula VIII (Pg₁=Boc):

6.13% w/w aqueous sodium hydroxide solution (184.1 g) was added to (R)-methionine (25.0 g; 0.166 mol) and tert-butanol (92.5 ml) was added. The mixture was cooled to 0-5° C. and di-tert-butyl dicarbonate (42.2 g) added in four portions over 45 minutes. The reaction mixture was warmed to 20° C. and stirred for 4 hours. The mixture was cooled to 0-5° C. and 30% w/w aqueous citric acid solution (164 g) was added, maintaining the temperature below 5° C. Dichloromethane (250 ml) was added and the mixture stirred at 20° C. for 15 minutes. The upper aqueous phase was separated and the organic phase retained. The aqueous phase was extracted with dichloromethane (125 ml) and the extract was combined with the retained organic phase. The combined organic phase was washed with water (250 ml) and then with 17% w/w aqueous sodium chloride (300 g). The organic phase was distilled at atmospheric pressure until a volume of 250 ml remained. The solution (which contains Boc-(R)-methionine) was cooled to −5 to 0° C. and N-methyl-morpholine (35.7 g) added maintaining the temperature −5 to 0° C. (S)-Alanine methyl ester hydrochloride (25.8 g) was added, followed by 1-hydroxybenzotriazole hydrate (24.7 g). 1-(3-Dimethylaminopropyl)-3-ethylcarbodiimide hydrochloride (36.1 g) was added in four portions over 1 hour. The mixture was stirred at −5 to 0° C. for 5 hours. Water (100 ml) was added maintaining the temperature below 5° C., and the mixture was stirred for 15 minutes. The organic phase was separated and washed successively with water (100 ml), 30% w/w aqueous citric acid solution (132 g), 9.1% w/w aqueous sodium bicarbonate solution (110 g) and 16.7% w/w aqueous sodium chloride (120 g). Dichloromethane (450 ml) was added to the organic phase and the mixture distilled at atmospheric pressure until a volume of 500 ml remained. The mixture (which contains Boc-(R)-Met-(S)-Ala-OMe) was cooled to 0-5° C. and trimethyloxonium tetrafluoroborate (24.7 g) was added in one portion keeping the temperature at 0-5° C. The mixture was allowed to warm to 20° C. over 30 minutes and then stirred for a further 4 hours. Potassium carbonate (96 g) was added in three portions and the mixture was reflux for 20 hours in total. Water (400 ml) was added maintaining the temperature <5° C. The mixture was stirred for 15 minutes at 20° C. and filtered through a celite pad (5.0 g). The lower organic phase of the filtrate was separated and washed with water (300 ml). The solution was distilled at atmospheric pressure until a volume of 120 ml remained. n-Butyl acetate (385 ml) was added and the solution was concentrated at under reduced pressure, 100 mbar, until a volume of 205 ml remained. The concentrate was cooled to 60° C. and isohexane (614 ml) was added maintaining the temperature ≧55° C. The mixture was cooled to 0° C. and stirred for 1 hour. The suspended crystalline solid was collected by filtration, washed with cold n-butyl acetate:isohexane (32 ml:101 ml), and then with cold isohexane (135 ml), then dried at 50° C. in a vacuum oven for 17 hours to give methyl (S)-2-[(R)-3-(N-[tert-butyloxycarbonyl]amino)-2-oxopyrrolidin-1-yl]propionate (23.7 g);

¹H NMR (d₆-DMSO): 1.30 (d, 3H), 1.40 (s, 9H), 1.80 (m, 1H), 2.25 (m, 1H), 3.20 (m, 1H), 3.30 (t, 1H), 3.65 (s, 3H), 4.10 (m, 1H), 4.65 (m, 1H), 7.10 (d, 1H)

The product (70 g) in water (595 ml) was cooled to 0-5° C. and lithium hydroxide monohydrate (11.15 g) in water (105 ml) was added over one hour with stirring. The reaction mixture was stirred for a further 2 hours at 0-5° C. and a solution of potassium hydrogen sulfate (37.84 g) in water (140 ml) was then added to the cold mixture over 2 hours. After a further hour the cold mixture was filtered. The collected solid was washed with a small volume of water and dried under vacuum at 40° C. to give (S)-2-[(R)-3-(N-[tert-butyloxycarbonyl]amino)-2-oxopyrrolidin-1-yl]propionic acid (21.8 g);

¹H NMR (d₆-DMSO): 1.33 (m, 3H), 1.79 (m, 1H), 2.27 (m, 1H), 3.28 (m, 2H), 4.06 (q, 1H), 4.55 (m, 1H), 7.09 (d, 1H).

Alternative Preparation of the Methyl (S)-2-[(R)-3-(N-[tert-butyloxycarbonyl]amino)-2-oxopyrrolidin-1-yl]propionate (Compound of Formula VIIIa (Pg₁=Boc, R=methyl))

Step 1.1 Boc Protection of D-Methionine

To D-Methionine (25.00 g, 0.168 mmol) was added 6.13% w/w sodium hydroxide solution (176 ml, 7.0 rel vol). t-Butanol (85.0 ml) was charged to the reaction mixture which was cooled to 4° C. Boc-anhydride (42.18 g, 183 mmol, 1.12 mol eq) was charged in four equal portions over 45 minutes, maintaining the batch temperature at less than 4° C. The reaction mixture was warmed to ambient (22° C.) and stirred overnight. The reaction mixture was cooled to 3° C. and charged with 30% w/w aqueous citric acid (49.05 g of citric acid, 1.52 mol eq, dissolved in 115 ml of water), maintaining the temperature below 5° C. Additional citric acid (15.00 g, 71.4 mmol) was charged to reduce the pH to less than 3. Dichloromethane (250 ml) was charged and the batch was warmed to ambient temperature (22° C.). After stirring for 15 minutes the batch was allowed to settle for 15 minutes. The lower organic layer was separated and retained. The aqueous layer was extracted with dichloromethane (125 ml). It was held at 20° C. for 15 minutes and allowed to settle for 15 minutes. The lower organic layer was separated and combined with the initial organic layer. Water (250 ml) was charged to the combined organic phase. The mixture was stirred at 20° C. for 15 minutes and then allowed to settle for 15 minutes. The lower organic phase was separated and brine (50 g of sodium chloride, 2 rel wt, dissolved in 250 ml of water, 250 g, 10.0 rel vol) was added. The batch was stirred at 20° C. for 15 minutes and allowed to settle for 15 minutes. The lower organic layer was separated and then concentrated from 440 ml to 250 ml by atmospheric distillation, with the bath at 62° C. Additional dichloromethane (4.00 ml) was charged and the organic phase was concentrated to 140 ml by atmospheric distillation. The moisture content was 0.06% w/w.

Step 1.2: Coupling of Boc-D-Methionine and Alanine Methyl Ester

The reaction mixture from step 1.1 was cooled to 4° C. and 4-methylmorpholine (38.8 ml 349 mmol 2.10 mol eq) was added evenly over 30 minutes, maintaining the temperature <5° C. Alanine methyl ester hydrochloride (25.80 g, 183 mmol, 1.10 mol eq) was charged, followed by HOBt.H₂O (24.71 g, 161 mmol, 0.96 mol eq). EDCI.HCl (36.07 g, 188 mmol, 1.12 mol eq) was charged in four equal portions over 1 hour, maintaining the temperature <5° C. The reaction mixture was stirred at <5° C. for around 5 hours and allowed to warm slowly to 20° C. overnight. After cooling to 4° C., water (100 ml) was charged. The mixture was stirred for 15 minutes at <10° C. and allowed to settle for 15 minutes. The lower organic layer was separated and water (100 ml) was added maintaining the temperature at <10° C. The mixture was stirred for 15 minutes and allowed to settle for 15 minutes. The lower organic layer was separated and charged with 30% w/w aqueous citric acid (38.50 g of citric acid, 1.20 mol eq, dissolved in 93 ml of water), maintaining the temperature at <10° C. The reaction mixture was stirred for 15 minutes and allowed to settle for 15 minutes. The lower organic layer was separated and charged with 9.10% w/w aqueous sodium hydrogen carbonate (10.0 g of sodium hydrogen carbonate, 0.71 mol eq, made up with 100 ml of water), maintaining the temperature at <10° C. The reaction mixture was stirred for 15 minutes at <10° C. and allowed to settle for 15 minutes. The lower organic layer was separated and charged with 16.7% w/w brine (20.00 g of sodium chloride dissolved in 100 ml of water). The reaction mixture was stirred for 15 minutes at <10° C. and allowed to settle for 15 minutes. The lower organic layer was separated, dichloromethane (450 ml) was added and concentrated to 430 ml by atmospheric distillation The water level was 0.05% w/w.

Step 1.3: S-Methylation of Boc-D-Met-Ala-OMe

To the reaction solution from step 1.2 was added dichloromethane (70 ml), half of this solution was carried forward. The flask was argon purged and dimethyl sulfate (7.9 ml 82.5mmols, 1.20 mol eq based on Dipeptide) was charged. The reaction mixture was heated to reflux (42° C.) and stirred for 27 hours.

Step 1.4: Cyclisation

A reaction flask containing the reaction mixture from step 1.3 was connected to a reversed Dean and Stark apparatus and a bleach trap. Potassium carbonate (19.02 g, 138 mmol, 2.0 mol eq) was charged producing a slurry. The reaction mixture was heated to reflux (42° C.) and charged with additional potassium carbonate (9.51 g, 69 mmol 1.0 mol eq) after 4.25 and 20 hr. The reaction mixture was cooled to 3° C. and water (200 ml) was added maintaining the temperature below 5° C. The reaction mixture was warmed to 20° C., stirred for 15 minutes and allowed to settle 15 minutes. The lower organic layer was separated and water (150 mL 3.3 rel vol) added. After stirring at 20° C. for 15 minutes and being allowed to settle for 15 minutes, the lower organic layer was separated and retained.

Step 1.5: Crystallisation and Isolation

n-Butyl acetate (176 ml) was charged to the organic solution from step 1.4 and the organic phase was concentrated to 90 ml by high vacuum distillation (bath temperature 75° C., pressure <100 mbar). iso-Hexane (282 ml) was charged keeping the temperature >45° C. Some white solid was formed which virtually all dissolved when the reaction mixture was heated to reflux (62° C.) The batch was cooled to 50° C. over 20 minutes and held at 50° C. for 30 minutes to give a suspension of the title product in crystalline form. The batch was cooled to 4° C. over 30 minutes and the slurry was filtered and allowed to deliquor. The product cake was displacement washed with a pre-chilled (4° C.) solution of n-butyl acetate and iso-hexane (19 ml:51 ml) and washed with iso-hexane (68 ml). The product was dried in a vacuum oven at 50° C. to give methyl (S)-2-[(R)-3-(N-[tert-butyloxycarbonyl]amino)-2-oxopyrrolidin-1-yl]propionate (yield @ 100% strength=16.01 g (67%)).

The product was analysed by HPLC and ¹H-NMR.

### EXAMPLE 4

### (Preparation of Compound of Formula IX (Pg=nitro; Pg₂=Boc) from Compound of Formula X (Pg=nitro; Pg₂=Boc; R=methyl))

A mixture of tert-butyloxycarbonyl-(S)-alanyl-(S)-arginyl(NO₂)-(S)-alanine methyl ester (formula X, Pg=nitro, Pg₂=Boc, R=methyl) (64.9 g) and acetonitrile (380 ml) was cooled to 16° C. and 1.0N sodium hydroxide solution (146.5 ml) was added over 2 minutes. The mixture was maintained at 9-12° C. for 90 minutes than allowed to warm to 18° C. and 1.0M sodium hydrogen sulfate solution (195 ml) was added. Solid sodium chloride (64.9 g) was added and the organic layer separated and the aqueous phase washed with acetonitrile. The organic phases were combined, DMF (40 ml) was added and the mixture cooled to 0-5° C. 4-Aminophenylacetamide (19.95 g) and 1-hydroxybenzotriazole monohydrate (12.83 g), followed by 1-(3-dimethylaminopropyl)-3-ethylcarbodiimide hydrochloride (28.0 g) were then added. The mixture was stirred with cooling for 16 hours and then allowed to warm up to ambient temperature. The reaction mixture was concentrated to give an oil. Isobutanol (420 ml) was added and the solution was filtered. 10% Aqueous citric acid solution (195 ml) was added to the filtrate, followed by solid sodium chloride (50 g). The organic phase was separated and washed with saturated sodium chloride solution and distilled until 105 ml of distillate was collected. The mixture was allowed to cool to ambient temperature and the solid was collected by filtration and dried under vacuum at 45° C. to give tert-butyloxycarbonyl-(S)-alanyl-(S)-arginyl(NO₂)-(S)-alanyl-4-aminophenylacetamide (formula IX, Pg₂=Boc) (65.35 g);

¹H NMR (d₆-DMSO): 1.23 (d, 3H), 1.38 (d, 3H), 1.45 (s, 9H), 1.59 (m, 3H), 1.77 (m, 1H), 3.22 (m, 2H), 3.37 (s, 2H), 4.03 (m, 1H), 4.36 (m, 1H), 4.46 (m, 1H), 7.24 (m, 2H), 7.58 (m, 2H).

Preparation of 4-Aminophenylacetamide:

(i) 4-Aminophenylacetic acid (22.0 g) was added portionwise over 15 minutes with stirring to a cold 2.0-2.5M solution of anhydrous hydrogen chloride in methanol (132 ml), maintaining the temperature of the mixture at 0-10° C. The mixture was then refluxed for one hour and concentrated by distillation at atmospheric pressure, collecting 72.6 ml of distillate. The mixture was then cooled to 50° C. and methyl tert-butyl ether (176 ml) added maintaining the temperature above 35° C. The mixture was then gradually cooled to 2.5° C. and held at this temperature for 1 hour. The resultant crystalline product was isolated by filtration and washed with methyl tert-butyl ether (2×20 ml) and dried under vacuum at 50° C. to give methyl 4-aminophenylacetate hydrochloride (28 g);

¹H NMR (d₆-DMSO): 3.60 (3H), 3.70 (2H), 7.36 (4H).

(ii) Methyl 4-aminophenylacetate hydrochloride (28 g) was added portionwise over 15 minutes with stirring to cold aqueous ammonia (density 0.91 g/ml; 84 ml), maintaining the temperature of the mixture at 15-25° C. The mixture was then stirred for 16 hours at ambient temperature. The mixture was cooled to 0-5° C. and held at this temperature for one hour. The resultant crystalline product was isolated by filtration, washed successively with water and acetonitrile, and dried under vacuum at 50° C. to give 4-aminophenylacetamide (16.7 g);

¹H NMR (d₆-DMSO): 3.16 (2H), 4.85 (2H), 6.49 (2H), 6.90 (2H).

### EXAMPLE 5

### (Preparation of Compound of Formula III Pg=nitro) from Compound of Formula VI (Pg=nitro; R=methyl)

1.0M Sodium hydroxide solution (180 ml) was added to 5-phenylpentanoyl-(S)-alanyl-(S)-arginyl(NO₂)-(S)-alanine methyl ester (formula VI, Pg=nitro; R=methyl) (17.75 g) in acetonitrile (180 ml) with stirring over ten minutes. The mixture was stirred for 3 hours, cooled to 0-5° C. and concentrated hydrochloric acid (3.5 ml) was added slowly to adjust the pH of the mixture to 2-3. The mixture was allowed to come to ambient temperature, then heated to gentle reflux and allowed to cool to 50° C. The organic phase was separated and volatile material removed by distillation, adding acetonitrile at intervals, until the still head temperature was 81° C. The reaction mixture was allowed to cool to ambient temperature over 2 hours with stirring. The mixture was cooled to 10° C. and the precipitated solid collected by filtration, washed with acetonitrile (2×15 ml) and dried under vacuum at 45° C. A mixture of the solid (21.8 g), water (100 ml) and acetonitrile (100 ml) was heated to reflux and allowed to cool slowly. The mixture was cooled to 14° C. and the precipitated solid collected by filtration, washed with acetonitrile (2×15 ml) and dried to give 5-phenylpentanoyl-(S)-alanyl-(S)-arginyl(NO₂)-(S)-alanine (17.3 g);

¹H NMR (d₆-DMSO): 1.18 (d, 3H), 1.27 (d, 3H), 1.52 (m, 7H), 1.70 (m, 1H), 2.13 (t, 2H), 2.55 (t, 2H), 3.13 (m, 2H), 4.18 (m, 1H), 4.26 (m, 2H), 7.17 (m, 3H), 7.27 (m, 2H).

### EXAMPLE 6

### (Preparation of Compound of Formula VI (Pg=nitro; R=methyl) from Compound of Formula X (Pg=nitro; Pg₂=Boc; R=methyl))

A mixture of tert-butyloxycarbonyl-(S)-alanyl-(S)-arginyl(NO₂)-(S)-alanine methyl ester (formula X, Pg₂=Boc; Pg=nitro; R=methyl) (79.6 g) in dichloromethane (668 ml) was cooled to 0-5° C. with stirring and anhydrous hydrogen chloride was bubbled through the mixture for 90 minutes. The mixture was then allowed to warm to ambient temperature. The mixture was then purged with argon for 90 minutes. The mixture was cooled to 0-5° C. and N-methylmorpholine (101.4 ml), 5-phenylpentanoic acid (27.4 g) and 1-hydroxybenzotriazole monohydrate (27.2 g) were successively added with stirring.

1-(3-Dimethylaminopropyl)-3-ethylcarbodiimide hydrochloride (32.4 g) was then added and the temperature of the mixture maintained at 0-5° C. for 15 minutes, then allowed to rise to ambient temperature. The mixture was allowed to stir for 16 hours, isobutanol (417 ml) added and the mixture washed with 1.0M sodium hydrogen sulfate solution (3×230 ml) and sodium carbonate solution (2×210 ml) and saturated sodium chloride solution (4×200 ml). The organic phase was concentrated, ethyl acetate (550 ml) was added and the mixture again concentrated. Additional ethyl acetate (550 ml) was added and the mixture was warmed and then allowed to cool, when a solid crystallised. The mixture was cooled in an ice-water bath and the crystalline solid isolated by filtration, washed with ethyl acetate (2×100 ml) and dried under vacuum at 50° C. to give 5-phenylpentanoyl-(S)-alanyl-(S)-arginyl(NO₂)-(S)-alanine methyl ester (formula VI, R=methyl) (75.5 g);

¹H NMR (d₆-DMSO): 1.18 (d, 3H), 1.28 (d, 3H), 1.52 (m, 7H), 1.69 (m, 1H), 2.13 (t, 2H), 2.55 (t, 2H), 3.16 (m, 2H), 3.62 (s, 3H), 4.26 (m, 3H), 7.17 (m, 3H), 7.27 (m, 2H).

Preparation of tert-butyloxycarbonyl-(S)-alanyl-(S)-arginyl(NO₂)-(S)-alanine Methyl Ester (Formula X (Pg₂=Boc; R=methyl)):

N-methylmorpholine (34.09 g) was added to a mixture of tert-butyloxycarbonyl-(S)-arginyl(NO₂)-OH (61.98 g), alanine methyl ester hydrochloride (20.93 g) and 1-hydroxybenzotriazole hydrate (10.13 g) in dichloromethane (750 ml) with stirring. The mixture was cooled to 0-5° C. and 1-(3-dimethylaminopropyl)-3-ethylcarbodiimide hydrochloride (36.0 g) was added. The mixture was stirred at 0-5° C. for 3 hours then at 22° C. for 30 minutes. The mixture was then cooled to 0-5° C. and anhydrous hydrogen chloride was then bubbled through the mixture keeping the temperature below 15° C. After 50 minutes, the mixture was warmed to 22° C. and purged with nitrogen. The mixture was cooled to 0-5° C. and N-methyl morpholine (92.9 g) was then added with stirring over 10 minutes maintaining the temperature of the reaction mixture below 20° C. The reaction mixture was cooled to 10° C. and tert-butyloxycarbonyl-(S)-alanine (28.35 g) was then added, followed by 1-(3-dimethylaminopropyl)-3-ethylcarbodiimide hydrochloride (28.8 g). The mixture was stirred for 15 hours and then filtered, washing the filter cake with dichloromethane (50 ml). The filtrate was washed with 1M sodium hydrogen sulfate (3×150 ml), followed by sodium carbonate solution, dried over anhydrous magnesium sulfate and concentrated by evaporation to give a pale yellow solid. The solid was dissolved in acetonitrile (169 ml) by heating to reflux and the solution allowed to cool The product which crystallised was collected by filtration and washed with cold acetonitrile (2×55 ml) to give tert-butyloxycarbonyl--(S)-alanyl-(S)-arginyl(NO₂)-(S)-alanine methyl ester (59.3 g);

¹H NMR (d₆-DMSO): 1.17 (d, 3H), 1.29 (d, 3H), 1.37 (s, 9H), 1.52 (m, 3H), 1.68 (m, 1H), 3.17 (m, 2H), 3.62 (s, 3H), 3.97 (m, 1H), 4.28 (m, 2H).

### EXAMPLE 7

### (Preparation of Compound of Formula V (Pg₁=Boc; Pg=nitro; R¹=benzyl) from Compound of Formula XIII (Pg₁=Boc; Pg=nitro; R=methyl) via Compound of Formula XI (Pg₁=Boc; Pg=nitro))

(1) 1M Sodium hydroxide solution (20 ml) was added in one portion to a slurry of the compound of formula XIII (Pg₁=Boc; Pg=nitro; R=methyl) (1.0 g) in acetonitrile (15 ml) and the mixture was stirred for 16 hours. Solid sodium chloride (0.5 g) was added in one portion and the mixture was acidified to pH 2 by dropwise addition of 1M hydrochloric acid (2.4 ml). Acetonitrile (5 ml) was added and the organic phase was separated. The aqueous phase was extracted with acetonitrile (2×10 ml) and the organic phases were combined to give a solution of the compound of formula XI (Pg₁=Boc; Pg=nitro). Hydroxybenzotriazole monohydrate (0.236 g) was added in one portion and the mixture was cooled to 0-5° C.

(2) A 4M solution of hydrogen chloride in dioxan (7 ml) was added in one portion to N-benzyl-4-(tert-butoxycarbonylamino)phenylacetamide (0.56 g) and the mixture was stirred for 2 hours. The mixture was concentrated and the residue maintained under vacuum for 2 hours. Acetonitrile (2 ml) was added to the residue and the slurry was cooled to 0-5° C. N-Methylmorpholine (0.8 ml) was added dropwise over one minute and the mixture stirred for 5 minutes.

(3) The cold mixture from step (1) was added to the mixture of step (2) and water (0.5 ml) was added to give a complete solution. 1-(3-Dimethylaminopropyl)-3-ethylcarbodiimide hydrochloride (0.465 g) was added in one portion and the resulting mixture stirred at 0-5° C. for 2 hours and then at ambient temperature for 16 hours. Potassium carbonate solution (10% w/w; 20 ml) was added and the organic phase was separated. The aqueous phase was extracted with acetonitrile (2×10 ml) and the combined organic phases were washed with saturated sodium chloride solution (20 ml) and then concentrated to give a pale yellow foam which solidified on standing. There was thus obtained the compound of formula V (Pg₁=Boc; Pg=nitro; R¹=benzyl) (1.07 g).

Preparation of the Compound of Formula XIII (Pg₁=Boc; Pg=nitro; R=methyl):

The compound of formula XIII (Pg₁=Boc; Pg=nitro; R=methyl) was obtained using an analogous procedure to that described in Example 6 from the compound of formula X (, but using the compound of formula VIII (Pg₁=Boc) in place of 5-phenylpentanoic acid. The compound of formula X (Pg₂=Boc; Pg=nitro; R=methyl) (10.81 g) was deprotected using anhydrous hydrogen chloride in dichloromethane. The mixture was purged with nitrogen for 16 hours and the resulting solid was slurried in acetonitrile (300 ml). The mixture was cooled to 0-5° C. and N-methylmorpholine (8 ml) was added dropwise over one minute and stirring was continued for 30 minutes. 1-Hydroxybenzotrazole monohydrate (2.84 g) and the compound of formula VIII (Pg₁=Boc) (6.89 g) were added and the mixture was stirred for 5 minutes. 1-(3-Dimethylaminopropyl)-3-ethylcarbodiimide hydrochloride (4.43 g) was added and the resulting mixture was stirred at 0-5° C. for 2 hours and then at ambient temperature for 12 hours. Aqueous potassium carbonate (10% w/v; 300 ml) was added and the mixture was stirred for 10 minutes. The aqueous layer was extracted with acetonitrile (2×10 ml) and the combined organic phases were washed with saturated sodium chloride solution (100 ml) and then concentrated to about 50 ml. Acetonitrile (100 ml) was added and the mixture was heated to reflux and allowed to cool to ambient temperature. The mixture was filtered under reduced pressure and the filter cake was washed with acetonitrile (2×30 ml). The solid was dried at 40° C. under vacuum to give the compound of formula XIII (Pg₁=Boc; Pg=nitro; R=methyl) (10.6 g).

Preparation of N-benzyl-4-(tert-butoxycarbonylamino)phenylacetamide

Triethylamine (6.67 ml) was added over two minutes to 4-(tertbutoxycarbonylamino)phenylacetic acid (10.0 g) and hydroxybenzotriazole monohydrate (0.236 g) in tetrahydrofuran 200ml). The mixture was cooled to 0-5° C. and 1-(3-dimethylaminopropyl)-3-ethylcarbodiimide hydrochloride (9.17 g) added. The mixture was stirred for three minutes and benzylamine (4.56ml) was added dropwise to the mixture. The resulting mixture was stirred at 0-5° C. for 1 hour and then at ambient temperature for 48 hours. Aqueous potassium carbonate (10% w/v, 200 ml) was added to the mixture, the separated aqueous layer was then extracted with THF (100 ml)and the combined organic extracts were washed with brine (2×100 ml) and then concentrated by vacuum distillation until approximately 50 ml of the organic extracts remained. Toluene (200 ml) was added and the resulting mixture was evaporated to dryness by vacuum distillation to leave a solid. Toluene (250 ml) was added to the solid and the mixture heated to reflux and allowed to cool to ambient temperature. The mixture was cooled in ice and then filtered. The isolated solid was washed with toluene (2×50 ml) and then dried at 40° C. under vacuum to give N-benzyl-4-(tert-butoxycarbonylamino)phenylacetamide (8.0 g).

### EXAMPLE 8

### Preparation of Compound of Formula IX (Pg=nitro; Pg₂=Boc) from Compound of Formula X (Pg=nitro; Pg₂=Boc; R=methyl) [Alternative Synthesis of tertbutyloxycarbonyl-(S)-alanyl-(S)-arginyl(NO₂)-(S)-alanyl-4-aminophenylacetamide (Formula IX, Pg₂=Boc)]

To a mixture of tert-butyloxycarbonyl-(S)-alanyl-(S)-arginyl(NO₂)-(S)-alanine methyl ester (formula X Pg=nitro; Pg₂=Boc; R=methyl) (101 g), water (25 ml) and tetrahydrofuran (905 ml) at 18° C. was added a solution of sodium hydroxide (9.57 g) in water (375 ml) over 10 minutes. The mixture was stood at ambient temperature for 18 hours. A solution of sodium hydrogen sulfate hydrate (42 g) in water (323 ml) was added. Sodium chloride (97 g) was added, the organic phase separated and cooled to 5° C. 4-Aminophenylacetamide (33.06 g), 1-hydroxybenzotriazole monohydrate (18.37 g) and 3-dimethylaminopropyl)-3-ethylcarbodiimide hydrochloride (44.6 g) were added The mixture was stirred at 5° C. for 40 minutes, 1-(3-dimethylaminopropyl)-3-ethylcarbodiimide hydrochloride (3.0 g) added and stirring continued at 10 to 16° C. for 2 hours. Sodium chloride (160 g) and water (650 ml) were added. The organic phase was separated. Tetrahydrofuran (450 ml) was added then 750 ml of distillate was collected by distillation at atmospheric pressure. Tetrahydrofuran (250 ml) and acetone (700 ml) were added at 60° C. The mixture was allowed to cool to ambient temperature then acetone (300 ml) was added. The solid was collected by filtration, washed with acetone (3×100 ml) and dried under vacuum at 42° C. to give tert-butyloxycarbonyl-(S)-alanyl-(S)-arginyl(NO₂)-(S)-alanyl-4-aminophenylacetamide (formula IX, Pg₂=Boc) (60.0 g);

¹H NMR (d₆-DMSO): 1.23 (d, 3H), 1.38 (d, 3H), 1.45 (s, 9H), 1.59 (m, 3H), 1.77 (m, 1H), 3.22 (m, 2H), 3.37 (s, 2H), 4.03 (m, 1H), 4.36 (m, 1H), 4.46 (m, 1H), 7.24 (m, 2H), 7.58 (m, 2H).

Preparation of 4-Aminophenylacetamide:

The 4-Aminophenylacetamide used in this example was obtained as follows:

(i) Methanol (200 ml) was charged to 4-aminophenylacetic acid (25.0 g). Sulfuric acid (18.0 ml) was added maintaining the temperature <20° C. The mixture was then refluxed for one hour and concentrated by distillation at atmospheric pressure until a volume of 135 ml. The mixture was then cooled to 50° C. and methyl tert-butyl ether (275 ml) added maintaining the temperature above 45° C. The mixture was then gradually cooled to 0-5° C. and held at this temperature for 1 hour. The resultant crystalline product was isolated by filtration and washed with cold methanol:methyl tert-butyl ether (20 ml:55 ml) and cold methyl tert-butyl ether (75 ml) then dried under vacuum at 45° C. to give methyl 4-aminophenylacetate hydrogensulfate (40.1 g);

¹H NMR (d₆-DMSO): 3.61 (s, 3H), 3.71 (s, 2H), 7.25 (m, 2H), 7.35 (m, 2H).

(ii) Methyl 4-aminophenylacetate hydrogensulfate (20 g) was added to 20% w/w aqueous sodium chloride (37.5 g). Aqueous ammonia (density 0.88 g/ml 50 ml) containing dissolved sodium chloride (7.5 g) was added maintaining the temperature 15-25° C. The mixture was then stirred for 16 hours at 22° C. The mixture was cooled to 0-5° C. and held at this temperature for one hour. The resultant crystalline product was isolated by filtration, washed with water (2×20 ml), and dried under vacuum at 45° C. to give 4-aminophenylacetamide (7.2 g);

¹H NMR (d₆-DMSO): 3.16 (2H), 4.85 (2H), 6.49 (211), 6.90 (2H).

### EXAMPLE 9

### (Preparation of Compound of Formula VI (Pg=nitro; R=methyl) from Compound of Formula X (Pg=nitro; Pg₂=Boc; R=methyl))

5-Phenylpentanoyl-(S)-alanyl-(S)-arginyl(NO₂)-(S)-alanine Methyl Ester (Formula VI, Pg=nitro; R=methyl) was Obtained as Follows:

Tert-butyloxycarbonyl-(S)-alanyl-(S)-arginyl(NO₂)-(S)-alanine methyl ester (60.0 g) was added to an anhydrous solution of benzenesulfonic acid (33.89 g) and dichloromethane (600 ml). Methanol (150 ml) was added and the agitated mixture was heated at reflux for 21 hours. The mixture was cooled to −5° C. and NMM (28.0 ml) added followed by 5-phenylpentanoic acid (30.26 g) and 1-hydroxybenzotriazole hydrate (9.89 g) maintaining the temperature at −5° C. A solution of 1-(3-dimethylaminopropyl)-3-ethylcarbodiimide hydrochloride (33.74 g) in methanol (60 ml) was added maintaining the temperature at −5° C. and then the mixture was stirred at −5° C. for 12 hours. The mixture was heated to 20° C. and washed with a solution of sodium chloride (10.3 g) in water (206 ml). The separated organic phase was washed with a solution of sodium chloride (10.3 g) in water (206 ml) and methanol (82 ml). The separated organic phase was heated to reflux and 350 ml of distillates collected. Acetonitrile (675 ml) was added and the solution heated to reflux and 400 ml of distillates were collected then the mixture allowed to cool and the crystalline solid isolated by filtration, washed with acetonitrile (70 ml) and dried under vacuum at 40° C. to give 5-phenylpentanoyl-(S)-alanyl-(S)-arginyl(NO₂)-(S)-alanine methyl ester (53.25 g);

¹H NMR (d₆-DMSO): 1.18 (d, 3H), 1.28 (d, 3H), 1.52 (m, 7H), 1.69 (m, 1H), 2.13 (t, 2H), 2.55 (t, 2H), 3.16 (m, 2H), 3.62 (s, 3H), 4.26 (m, 3H), 7.17 (m, 3H), 7.27 (m, 2H).

Preparation of tert-butyloxycarbonyl-(S)-alanyl-(S)-arginyl(NO₂)-(S)-alanine Methyl Ester (Formula X, Pg₂=Boc; Pg=nitro; R=methyl):

Tert-butyloxycarbonyl-(S)-arginyl(NO₂)-(S)-alanine methyl ester (formula XIV, Pg=nitro; Pg₃=Boc; R=methyl) (46.91 g) was added to an anhydrous solution of benzenesulfonic acid (22.15 g) and dichloromethane (750 ml). The agitated mixture was heated at reflux for 6 hours. The mixture was cooled to −5° C. and NMM (19.3 ml) added followed by tert-butyloxycarbonyl-(S)-alanine (21.95 g), 1-hydroxybenzotriazole hydrate (7.29 g) and 1-(3-dimethylaminopropyl)-3-ethylcarbodiimide hydrochloride (24.54 g) maintaining the temperature at −5° C. The mixture was stirred at −5° C. for 12 hours then heated to 20° C. and washed twice with a solution of sodium chloride (8.4 g) in water (168 ml). The separated organic phase was heated to reflux and 660 ml of distillates collected. Acetonitrile (540 ml) was added and the solution heated to reflux and 280 ml of distillates were collected then the mixture allowed to cool and the crystalline solid isolated by filtration, washed with acetonitrile (50 ml) and dried under vacuum at 50° C. to give tert-butyloxycarbonyl-(S)-alanyl-(S)arginyl(NO₂)-(S)-alanine methyl ester (45.09 g);

¹H NMR (d₆-DMSO): 1.17 (d, 3H), 1.29 (d, 3H), 1.37 (s, 9H), 1.52 (m, 3H), 1.68 (m, 1H), 3.17 (m, 2H), 3.62 (s, 3H), 3.97 (m, 1H), 4.28 (m, 2H).

Preparation of tert-butyloxycarbonyl-(S)-arginyl(NO₂)-(S)-alanine Methyl Ester (Formula (XIV), Pg₂=Boc; Pg=nitro; R=methyl):

An agitated mixture of alanine methyl ester hydrochloride (30.61 g) in dichloromethane (950 ml) was cooled to −5° C. and NMM (54.2 ml) added maintaining the temperature at −5° C. Tert-butyloxycarbonyl-(S)-arginyl(NO₂)-OH (formula XV, Pg=nitro, Pg₃=Boc) (70.0 g), 1-hydroxybenzotriazole hydrate (16.78 g) and 1-(3-dimethylaminopropyl)-3-ethylcarbodiimide hydrochloride (52.61 g) added maintaining the temperature at −5° C. The mixture was stirred at −5° C. for 12 hours then heated to 20° C. and n-butyl acetate (12.6 ml) added. The mixture was washed twice with water (225 ml) and the separated organic phase was heated to reflux until 600 ml of distillates were collected. n-Butyl acetate (700 ml) was added and the solution was distilled under reduced pressure until 530 ml of distillates were collected. The mixture was allowed to cool to 22° C. and the crystalline solid isolated by filtration, washed with n-butyl acetate (140 ml) and dried under vacuum at 40° C. to give tert-butyloxycarbonyl-(S)-arginyl(NO₂)-(S)-alanine methyl ester (80.1 g);

¹H NMR (d₆-DMSO): 1.29 (d, 3H), 1.38 (s, 9H), 1.52 (m, 3H), 1.63 (m, 1H), 3.15(m, 2H), 3.62 (s, 3H), 3.96 (m, 1H), 4.27 (m, 1H).

### EXAMPLE 10

### (Preparation of Compound of Formula I from Compound of Formula V (Pg₁=Boc; Pg=nitro; R¹=benzyl)

The compound of formula V (Pg₁=Boc; Pg=nitro; R¹=benzyl), prepared according to Example 7 may be converted into the compound of formula IV (Pg=nitro; R¹=benzyl) and coupled with the compound of formula III (Pg=nitro) using an analogous process to that described in Example 2. The resulting compound of formula II (Pg=nitro; R¹=benzyl) may then be converted into the salt of a compound of formula I by a catalytic hydrogenation in the present of a Pd/C catalyst using an analogous process to that described in Example 1.

1. A process for the manufacture of a salt of the compound of formula I: 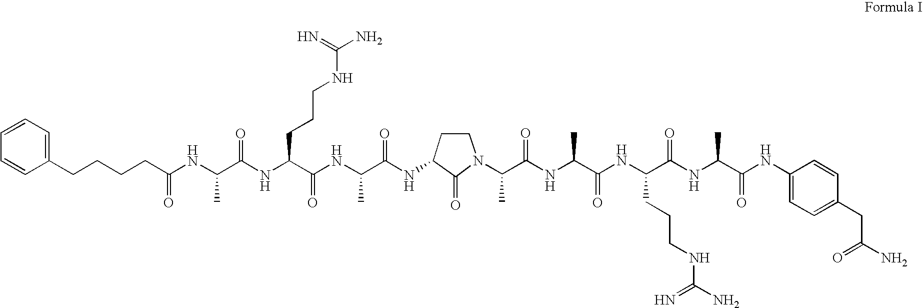
which comprises deprotection of a compound of the formula II or a salt thereof: 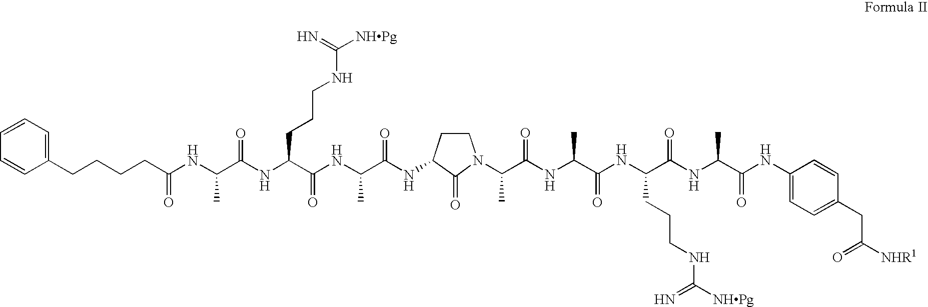
wherein:
each Pg is, independently, an arginine protecting group; and
R¹ is hydrogen or a protecting group for an amino group of an acetamide moiety.

2. A process according to claim 1 wherein the protecting groups Pg on the two arginyl residues are the same.

3. A process according to either claim 1 or claim 2 wherein both protecting groups Pg are nitro and R¹ is hydrogen.

4. A process according to claim 3 wherein the nitro groups protecting the arginyl residues in formula II are removed by chemical reduction.

5. A process according to claim 4 wherein the chemical reduction is a catalytic hydrogenation carried out in the presence of a solvent or mixture of solvents.

6. A process according to claim 5 wherein the catalytic hydrogenation is carried out in aqueous acetic acid containing a second acid which is stronger than acetic acid.

7. A process according to any one of the preceding claims wherein the compound of formula II or a salt thereof, is manufactured by a process which comprises coupling a carboxylic acid of the formula III or a salt thereof, 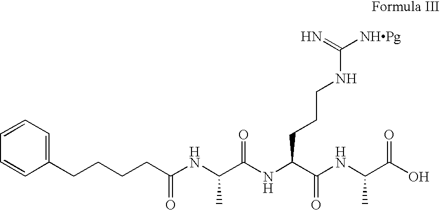
with an amine of the formula IV: 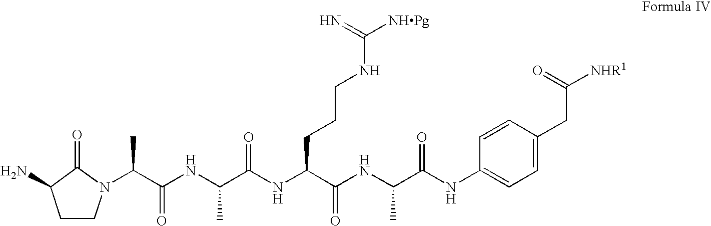
wherein R¹ and each Pg are as defined in claim 1.

8. A process according to claim 7 wherein the compound of formula IV is prepared by a process comprising removal of an amino protecting group Pg₁ from a compound of the formula V: 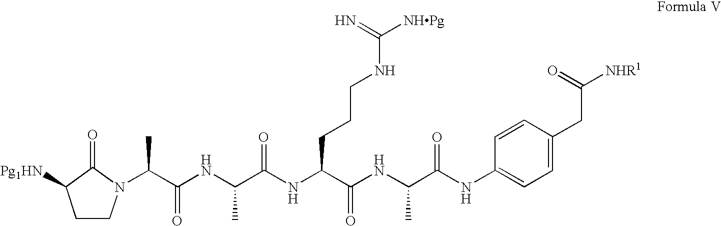
wherein:
Pg and R¹ are as defined in claim 1; and
Pg₁ is an amino protecting group which can be selectively removed in the presence of Pg and R¹ if the latter is other than hydrogen.

9. A process according to claim 8 wherein the compound of the formula V is prepared by a process comprising coupling a compound of the formula VII wherein Pg and R¹ are as defined in claim 1: 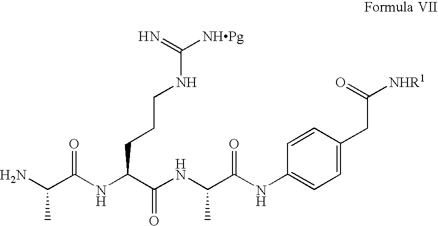
with a carboxylic acid of the formula VIII or a salt thereof: 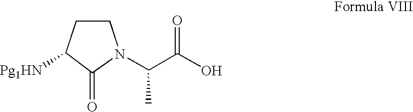
wherein Pg₁ is an amino protecting group.

10. A process according to claim 9 wherein the compound of the formula VII is prepared by a process comprising selectively removing the amino protecting group Pg₂ from a compound of formula IX: 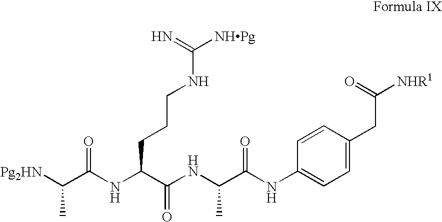
wherein:
Pg and R¹ are as defined in claim 9; and
Pg₂ is an amino protecting group which can selectively removed in the presence of Pg and R¹ if the latter is other than hydrogen.

11. A process according to claim 10 wherein the compound of the formula IX is prepared by a process comprising the steps:
(a) hydrolysis of the ester functionality of the compound of formula X to form a carboxylic acid group: 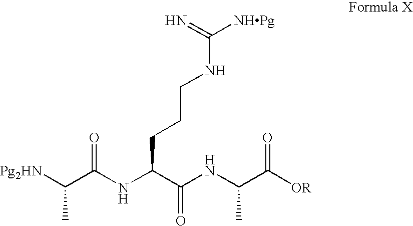
wherein Pg and Pg₂ are as defined in claim 10, and R is alkyl or aralkyl; and
(b) coupling the product of step (a) with a compound of formula XII: 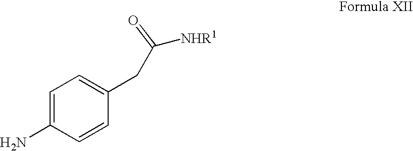
wherein R¹ is hydrogen or a protecting group for an amino group of an acetamide moiety.

12. A process according to claim 8 wherein the compound of the formula (V) is prepared by a process comprising coupling a compound of the formula XI 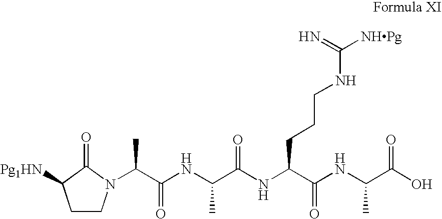
wherein Pg and Pg₁ are as defined in claim 8 with a compound of the formula XII 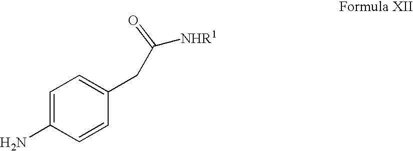
wherein R¹ is hydrogen or a protecting group.

13. A process according to claim 12 wherein the compound of the formula XI is prepared by a process comprising hydrolysis of the ester of the formula XIII: 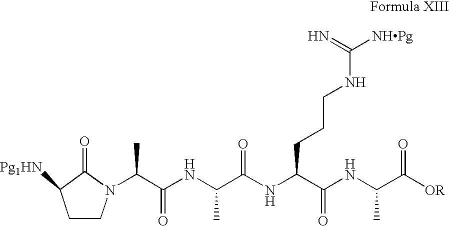
wherein:
R is alkyl or aralkyl;
Pg₁ is an amino protecting group which can be selectively removed in the presence of Pg and R¹ if the latter is other than hydrogen; and
Pg is an arginine protecting group.

14. A process according to claim 13 wherein the compound of the formula XIII is obtained from a compound of the formula X as defined in claim 11 by a process comprising the steps:
(a) removal of Pg₂ from the compound of formula X; and
(b) coupling the product of step (a) with a compound of the formula VIII as defined in claim 9.

15. A process according to claim 7 wherein the compound of the formula III or salt thereof is prepared by a process comprising hydrolysis of an ester of formula VI: 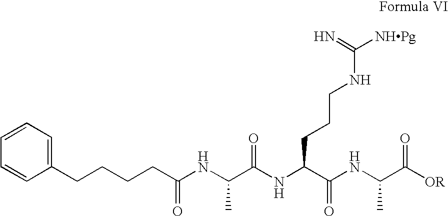
wherein:
R is alkyl or aralkyl; and
Pg is as defined in claim 7.

16. A process according to claim 15 wherein the compound of the formula VI is prepared by a process comprising the steps:
(a) removal of Pg₂ from a compound of formula X as defined in claim 11; and
(b) coupling the product of step (a) with 5-phenylpentanoic acid.

17. A process according to claim 7 wherein the compound of the formula III and the compound of the formula IV are both derived from a compound of the formula X as defined in claim 11.

18. The compound of the formula II as defined in claim 1.

19. The compound of the formula III as defined in claim 7.

20. The compound of the formula IV as defined in claim 7.

21. The compound of the formula V as defined in claim 8.

22. The compound of the formula VI as defined in claim 15.

23. The compound of the formula VII as defined in claim 9.

24. The compound (S)-2-[(R)-3-(N-[tert-butyloxycarbonyl]amino)-2-oxopyrrolidin-1-yl]propionic acid.

25. The compound of the formula IX as defined in claim 10.

26. The compound of the formula X as defined in claim 11.

27. The compound of the formula XI as defined in claim 12.

28. The compound of the Formula XIIa: 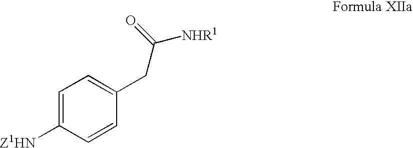
wherein R¹ is a protecting group for an amino group of an acetamide moiety; and
Z¹ is H or an amino protecting group.

29. The compound of the formula XIII as defined in claim 13.

30. A process for preparing a compound of the formula X as defined in claim 11 which comprises selective removal of Pg₃ from a compound of formula XIV by and coupling with a Pg₂ protected (S)-alanine: 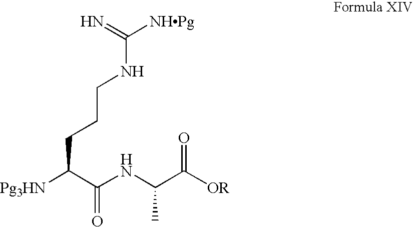
wherein
Pg₃ is an amino protecting group which can be selectively removed in the presence of Pg;
Pg₂ is an amino protecting group;
Pg is an arginine protecting group; and
R is alkyl or aralkyl.

31. A process according to claim 30 wherein the compound of the formula XIV is prepared by a process comprising coupling of a compound of the formula XV or a salt thereof, and a compound of the formula XVI or a salt thereof: 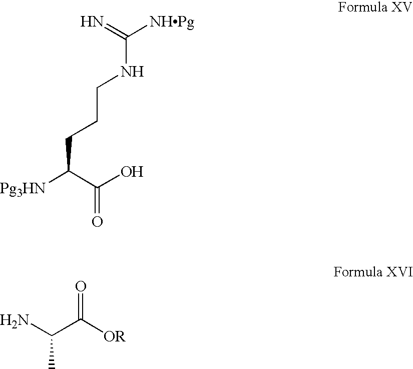
wherein Pg, Pg₃ and R are as defined in claim 30.

32. A process for the preparation of a compound of the formula VIII as defined in claim 9 comprising hydrolysis of the ester of the formula VIIIa 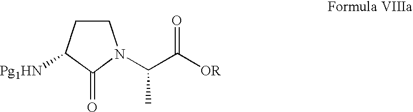
wherein Pg¹ is as defined in claim 9; and R is alkyl of aralkyl.

33. A process according to claim 32 wherein the hydrolysis is carried out under aqueous basic conditions using lithium hydroxide as the base.

34. A process for preparing of 4-aminophenylacetamide comprising the steps:
(i) esterification of 4-aminophenylacetic acid with a suitable alcohol in the presence of sulphuric acid to give a 4-aminophenylacetate ester hydrogensulphate salt; and
(ii) reacting the product of step (i) with ammonia.

35. A process for the manufacture of a compound of the formula II or a salt thereof as defined in claim 1 comprising coupling a carboxylic acid of the formula HI or a salt thereof, 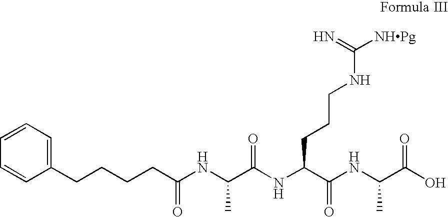
with an amine of the formula IV 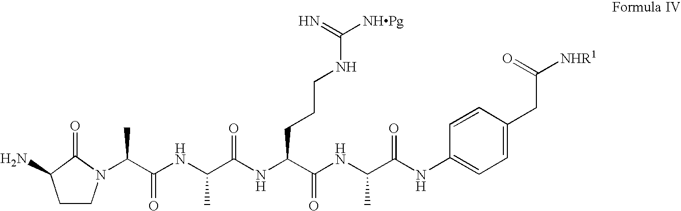
wherein Pg and R¹ are as defined in claim 1.
